(19) United States
(12) Patent Application Publication
(10) Pub. No.: US 2008/0058681 A1
(43) Pub. Date: Mar. 6, 2008

(54) Portable system for monitoring the position of a patient's head during videonystagmography tests (VNG) or electronystagmography (ENG)

(76) Inventors: Henry Eloy Sand Casali, Florida (AR); Miguel Angel Lacour Argerich, Capital Federal (AR); Marcos Maria Ledesma Williams, Pilar,Bs. As. (AR); Guillermo Horacio Diez, Mar del Plata, Bs. As. (AR)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/895,831

(22) Filed: Aug. 28, 2007

Related U.S. Application Data
(60) Provisional application No. 60/841,096, filed on Aug. 30, 2006.

(51) Int. Cl. A61B 3/00

(52) U.S. Cl. 600/587; 600/558

(57) ABSTRACT

System and method that include real time monitoring of the position and movement of the head in relation to a visual stimulator during videonystagmography (VNG) or electronystagmography (ENG) studies to a patient. Ultrasound sensors and transmitters combined with accelerometers or detectors of gravity are used, or other as necessary. The sensors signals are processed by a software that monitors position, facilitating the operator task and making a quality control of the diagnosis maneuvers providing a reproducible test method for the patients. Reproducibility is ensured by means of the acceptance limits provided by the software. The software also provides real time alarms that notice and guide the operator to make the maneuvers in a suitable and reproducible way. The invention provides a way to ensure reproducibility in the tests generating assistance to the operators which guides them throughout the process so that they can conduct the tests in the same way for all the patients. All in a portable system that allows its use in any medical office. The system is easy to move since it fits in a briefcase and is connected via external way to a “laptop”-type, portable or desktop computer.

### SCOPE OF THE INVENTION

This invention is under the scope of equipment employed in medicine, especially in relation to videonystagmography tests.

### STATE-OF-THE-ART

Videonystagmographers (VNG) and electronystagmographers (ENG) are equipment known for several years.

Their specific function is the diagnosis of equilibration, balance and vertigo problems of a person by testing the interrelation among the middle ear, the brain and the eye movements.

The diagnosis is based on the stimulation of the balance system, by means of a special positioning of the head, heating/cooling of the middle ear, head movements or visual stimulation of the patient and his response through eye movements, specially nystagmuses.

The different equipment manufacturers have developed techniques and software to stimulate and record responses both in analogical and digital form. Although some progress have been made in providing measurements of distances and/or speeds, there is still no system which can be consistent in its operation, therefore, the tests made by different technicians are not comparable. On the other hand, tests are not quality controlled.

The present results of the tests depend totally on the training, education and care of the technician or professional doing the test.

The dimensions of the equipment available in the market make it impossible to move them, limiting their use to medical doctor's offices, because they require permanent installation with mountings and fittings which prevent their mobility.

### BRIEF DESCRIPTION OF THE FIGURES

FIG. 1 shows a global block diagram of a preferred form of system performance for the present invention.

FIG. 2 shows a preferred form of system performance for the present invention giving details of the basic components.

FIG. 3 shows the different types of monitoring of the position and movements of the patient's head, made by the system according to this invention.

FIG. 4 shows a block diagram that displays the interaction between the operator and the patient during the VNG or ENG tests through the system of this invention.

FIG. 5 shows a main block diagram that gives details of the components of the system according to the present invention and its interrelation.

FIG. 6 shows a block diagram which describes in detail the registration process and the data transmission to the computer.

FIG. 7 shows a block diagram which describes the signaling process that the present invention system can make.

FIG. 8 shows a block diagram of the oculomotor or optokinetic tests that can be carried out with the system according to the present invention.

FIG. 9 shows a block diagram of the postural tests that can be carried out with the system according to the present invention.

FIG. 10 shows a block diagram of the caloric stimulation tests that can be carried out with the system according to the present invention.

FIG. 11 shows a block diagram of the rotatory stimulation tests (Active Head Rotatory (AHR)) that can be carried out with the system according to the present invention.

FIG. 12 shows the screen where the Software verifying the position of the patient opposite the visual stimulator in real time warns the operator to correct the operation as necessary.

FIG. 13 shows the screen where the Software verifying the angular position of the patient's head in real time warns the operator to correct the operation as necessary.

FIG. 14 shows the screen where the Software verifying in real time the position of the head respect to the visual stimulator and the angular motion of the patient's head with alarm (in red) of movements or positioning outside the reproducibility range, and which also monitors the eye movement in real time and makes a chart of the eye movement in horizontal and vertical channel, warns the operator to correct the operation as necessary.

### SUMMARY OF THE INVENTION

In order to correct the disadvantages of the state of the art, the inventors propose a portable system of detection in real time of the three-dimensional position and the speed of movement of a person's test attached to a test of videonystagmography, along with a software using this system of positioning to guide the technicians and professionals using the videonystagmography equipment to perform the different operations of positioning and/or rotation of the patient's head, thus ensuring that the tests are made in a correct, reproducible and consistent way.

The equipment is carried in a briefcase and connected to a portable or personal computer (PC) via external serial communication.

### DETAILED DESCRIPTION OF THE INVENTION

Dizziness and problems of balance, including vertigo, are one of the most common causes of complaints received by the medical professionals at present.

There exist many causes and abnormalities that produce these symptoms. These causes can be affecting the central nervous system or the peripheral one, but most of the problems derive from the inner ear organs responsible for the detection of the position and the movement of the head, and there can also exist problems related to the nerves that communicate the inner ear with the brain, vascular problems, blood flow in the spine, traumatisms, etc.

In the inner ear we find the semicircular channels, whose orthogonal space orientation allows the record of the position, speed and acceleration of the head. The information obtained by this organ generates automatic reflections and is in turn processed together with other somatosensorial and visual signals that the central nervous system gathers to obtain the balance, stability, or sensation of movement, speed up, etcetera.

The information is fed back to the eye muscles for its positioning through the vestibular ocular reflex (VOR) which is the one that permanently coordinates the relative position detected by the semicircular channels of the internal ear with the position of the eyes.

Diagnosis of problems in the system of detection of position and balance is based on the observation of the eye movements that respond to the VOR. In case of abnormal detection of position, speed or acceleration on the part of the semicircular channels or by the nervous system, the eyes will respond abnormally trying to adapt to an abnormal position, this will produce a movement of adaptation of the eye called nystagmus.

Nystagmuses are classified in normal and abnormal, the normal ones respond to real stimuli and true detected positions, and the abnormal ones respond to positions detected erroneously by the vestibular or nervous system and do not correspond to real positions, speeds or accelerations.

The detection and analysis of the nystagmus together with controlled stimuli, provide valuable information for the diagnosis of problems in the inner ear and/or central nervous system and/or the communication between both. The exploration of the vestibular system requires the VOR, the function of the cerebellum and the stability evaluation.

This involves a great number of tests to explore the different parts of the vestibular system and the neurological structures involved in the maintenance of the balance.

The videbnystagmography is part of the set of tests that allow the exploration of the vestibular system. This consists basically of four sub-groups of tests:
Oculomotor or Optokinetic (Saccades, Smooth Pursuit, OKN, Gaze and Spontaneous Nystagmus).
Positional and Positioning; DIX-Hallpike Maneuvers.
Caloric Stimulation.
Rotatory (Active Head Rotatory (AHR)).

All the tests are evaluated by recording the eye movements, whose particular case are the nistagmuses.

Each test is carried out by means of different stimulation techniques that allow the analysis of the responses of the different components of the vestibular system. These techniques or protocols differ greatly with each other, and display an important amount of variables to be controlled; which can be sources of error in the tests, their response being intrinsically dependant of the state of the variables to be controlled.

Due to the complexity and diversity of the tests to be taken, it is necessary to control a great number of variables, which implies the need for highly qualified personnel.

The aim of the portable system according to the present invention is to automatically monitor these variables to:
Standardize the conduction of the tests and, therefore, increase the reliability of the results.
Standardize protocols and generalize the analysis producing reliable common patterns that facilitate the creation of compatible data bases to obtain new conclusions of diagnosis.
Significantly reduce the curve of training of the personnel in charge of conducting the tests by means of an expert system capable of producing by means of audio and visual presentation the orders necessary to make the tests.
Ability to measure these parameters with a portable system, allowing the conduction of massive tests on patients.

The tests that can be conducted with the system of the present invention are:

Oculomotor or Optokinetic Test:

They are made by means of visual stimuli produced in a bar or monitor, called visual stimulator.

The patient must be seated, straight and with his head steady. Stimuli must be generated to known amplitudes or eccentricity degrees. This is achieved by placing the patient to a distance d from the screen:
d=eccentricity/tan(αdesired)

Since the technique is standardized, you must check that the patient's head does not move and the distance is adequate to produce the stimulation required for the different tests.

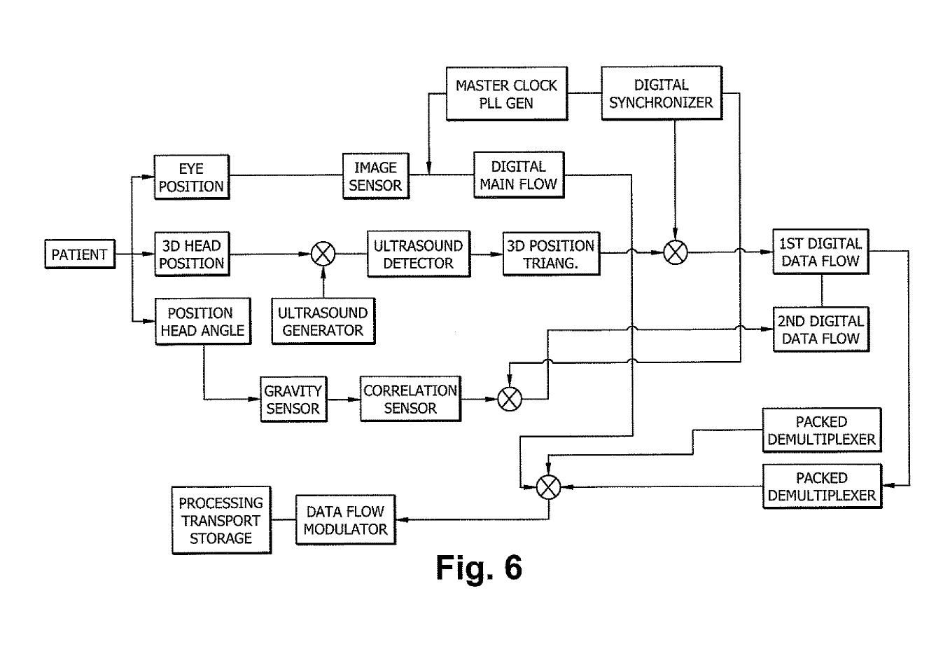

The block diagram in FIG. 6 schematically shows the operation of the oculomotor or optokinetic tests that are conducted with the system according to the present invention.

The visual stimulator produces different types of stimuli, a point that comes and goes horizontally or vertically with uniform speed or that always moves repeatedly towards the same direction, or a programmed stimulus.

The visual stimulus is caught by the eyes which send the signal to the brain which acts on the eye muscles to obtain its positioning thus producing the follow-up of the stimulus.

If the eyes follow the stimulus correctly then the feedback system works properly. In the case that the eyes do not follow the stimulus correctly or the patient reports dizziness, this is due to a fault in the feedback and/or interrelation between the eyes, the brain and its relation with the inner ear which will be expressed in abnormal nystagmuses, in connection with dizziness or other symptoms detected by the system.

Positional and Positioning Test or DIX-Hallpike Maneuvers:

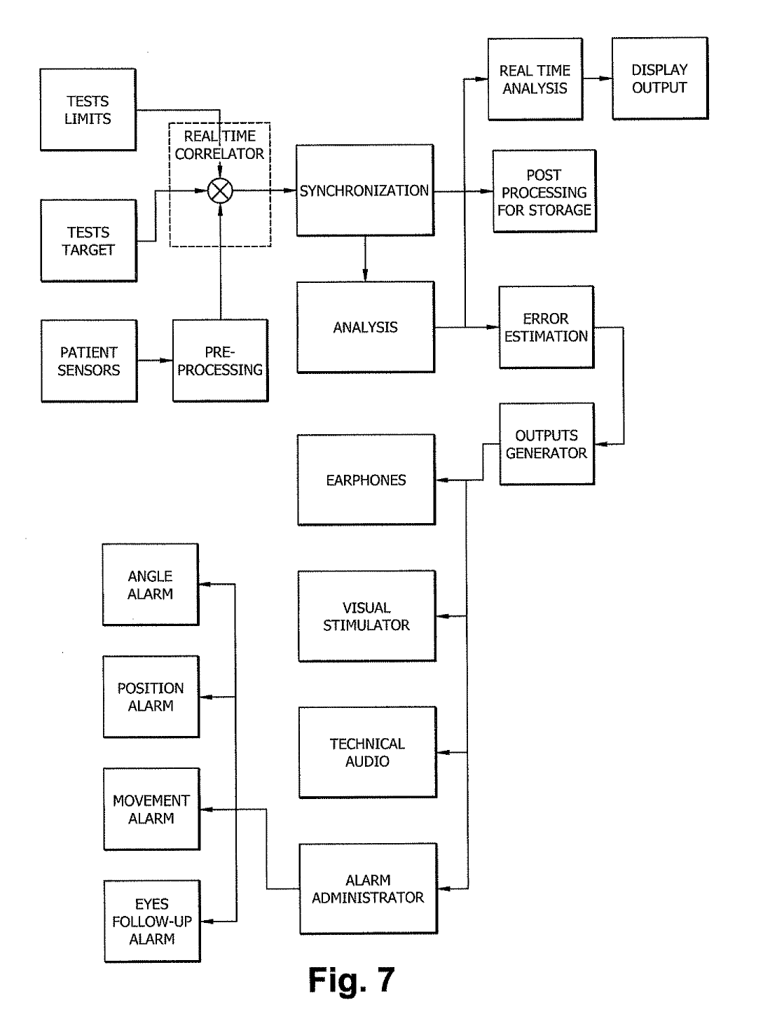

The block diagram in FIG. 7 schematically shows the operation of the Positional and Positioning tests that are conducted with the system according to the present invention.

In order to determine if the nistagmuses origin is postural (positional) or positional (positioning), it is required to explore the patient in certain positions.

The nystagmus that appears as a result of a movement of the head or the head and the body, is said to be positional nystagmus (positioning); however, if it occurs in certain position of the steady head, it is of postural origin (positional).

For example, being the patient sitting the head is turned approximately 90° to the right and is, abruptly taken to supine position with the turned head; after a few seconds it is returned to the starting point. The maneuver is repeated but with the head turned about 90° towards the left.

In the case of postural tests, the patient takes certain positions of the head with the aid of the operator. These positions are detected by the semicircular channels of the inner ear and, by being processed by the brain, they make the eyes accommodate to the detected position.

If some problem occurs in this process, the output of the eye movement will not have correlation with the stimulus position and the eyes will look for unreal positions, that generate abnormal nystagmuses.

Caloric Stimulation Test.

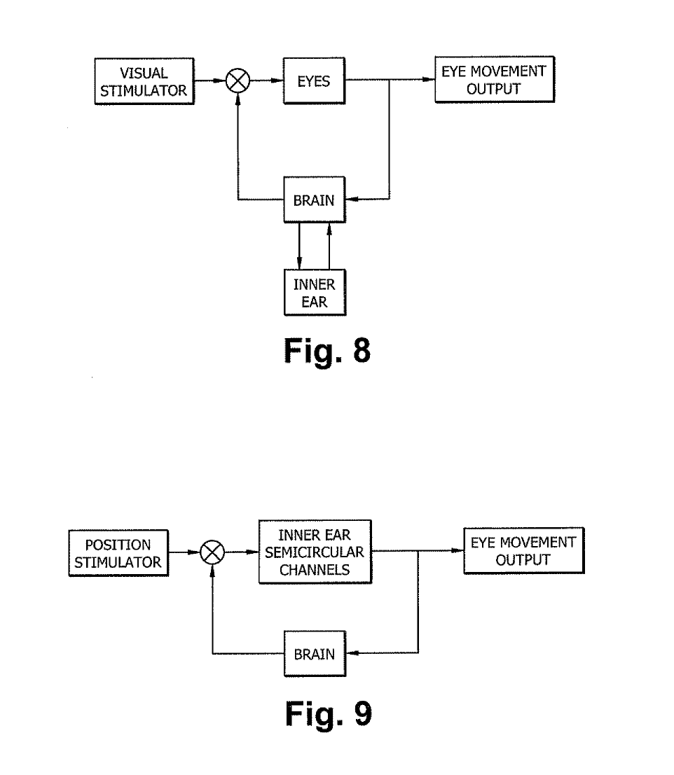

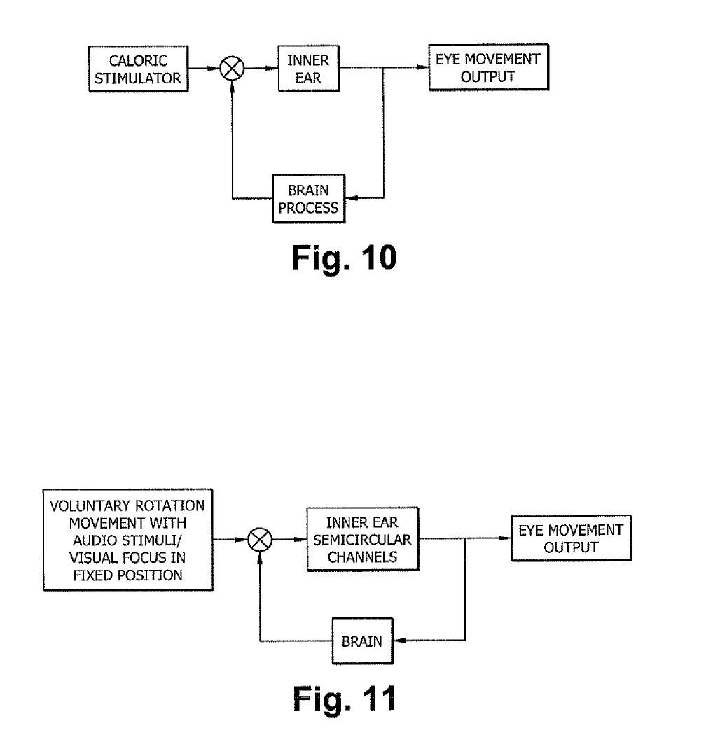

The block diagram in FIG. 8 schematically shows the operation of the caloric stimulation tests that are conducted with the system according to the present invention.

The caloric tests are based on the stimulation of the external channel of the ear by means of thermal changes with hot or cold air or water.

Heat or coldness are transmitted through the tympanic membrane and the middle ear, reaching the inner ear, particularly the semicircular channels that, when varying their temperature, vary their nervous response, mainly due to the change that the temperature produces in the internal liquid of the channels. When the viscosity of this liquid varies based on the temperature, its mobility varies too and, therefore, the stimulus that it produces inside the channels. When the patient gets dizzy because of the stimulus, the brain tries to compensate the eye position according to the information received from the semicircular channels. When these signals are altered by the caloric stimulation, nystagmus takes place whose normality or abnormality will depend on the degree of the stimulus received.

The optimal position for the stimulation of the semicircular channels involves the patient lying down with his head at about 30° over the horizontal position. In this way the semicircular channel takes a vertical position.

Rotatory Test (Active Head Rotation (AHR))

The block diagram in FIG. 9 schematically shows the operation of the rotatory stimulation tests that are conducted with the system according to the present invention.

It is based on the use of absolutely physiological rotatory stimuli, represented by voluntary turns of the head keeping the look fixed at a special target point. The individual must make a sequence of movements to left and right with increasing frequency indicated by the stimulation system.

When making the voluntary rotatory movements, the inner ear detects them and notifies the brain which sends compensation signals to the eyes.

The eye movement analysis determines if the correction measured by the ear and processed by the brain is normal, or responds to pathological parameters.

Description of the System

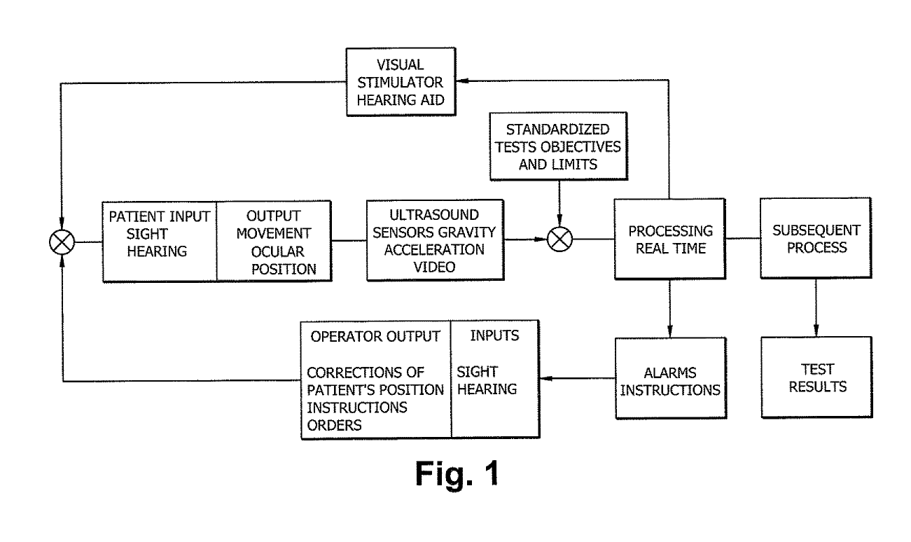

FIG. 1 shows a general block diagram of the system according to the present invention where the interrelation between the patient and the operator can be observed.

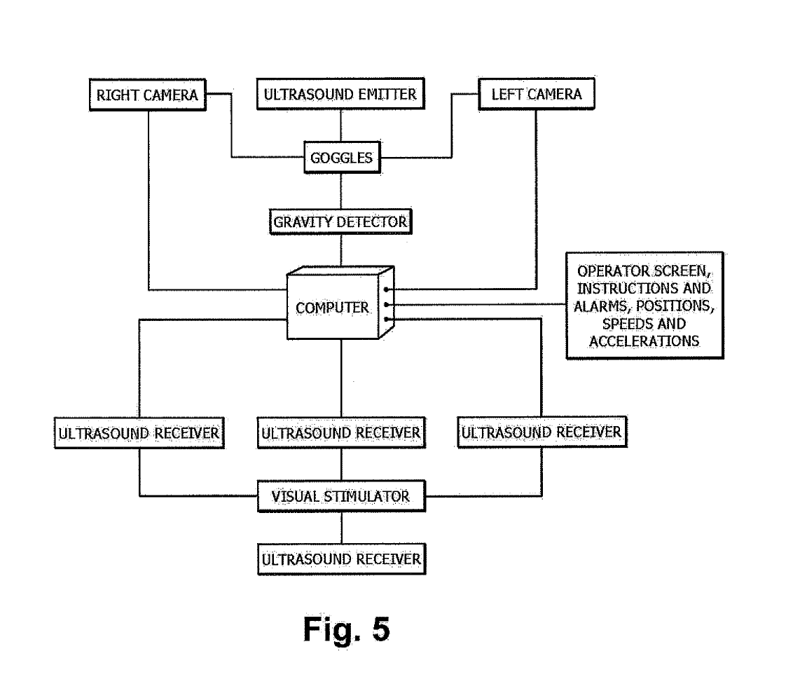

In FIG. 5 we can observe a main block diagram that gives details of the components of the system according to the present invention and its interrelation.

Specifically, the portable system for the monitoring of the patient's head positioning during videonystagmography (VNG) and/or electronystagmography (ENG) studies according to this invention allows determining the position of the patient's head in relation to a visual stimulator placed at the front of his head, the patient's head inclination in relation to his vertical axis (normal) and the patient's head rotation in relation to his vertical axis (normal), where the system re-feeds these determinations of the patient's head position in the space to the operator and/or patient, with the objective of keeping the VNG or ENG studies within a defined standard and thus guaranteeing the reproducibility of the said studies.

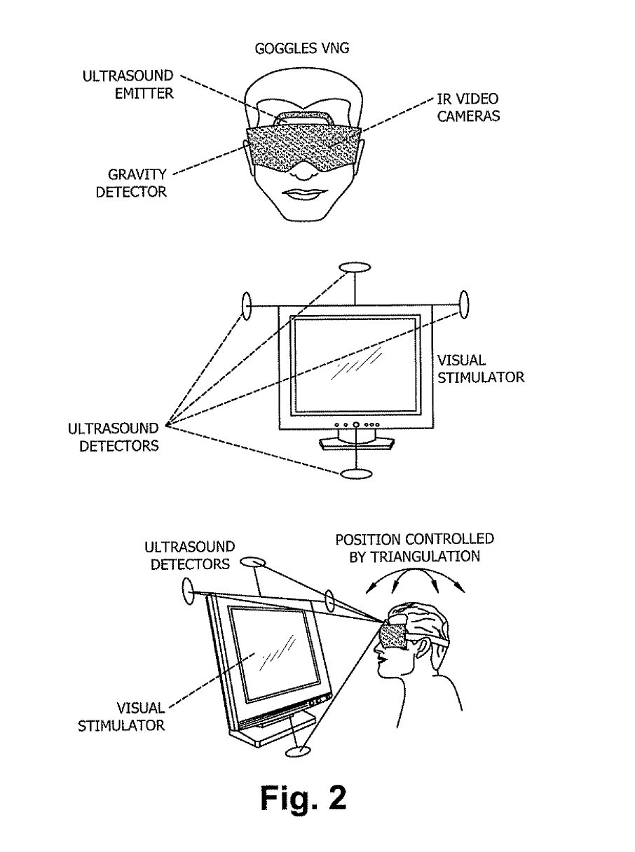

According to FIG. 2, a preferred way of realization the system of the present invention includes eyeglasses and a visual stimulator connected to a personal computer (PC).

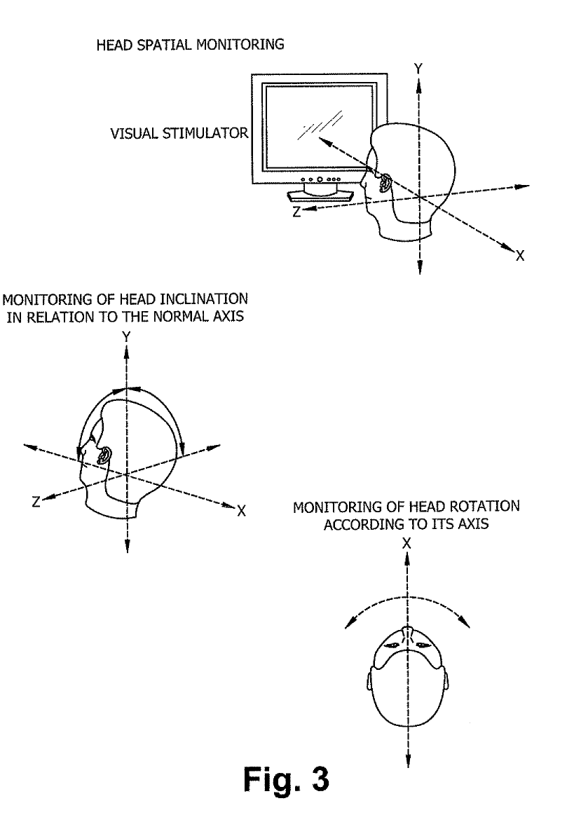

FIG. 3 shows the different types of monitoring of the position and movements of the patient's head, made by the system according to this invention, whose determinations allow the standardization of the tests making it possible the necessary reproducibility.

Indeed, the system according to this invention determines and monitors the position and movement of the patient's head in real time. This monitoring can be divided into three different controls:
1) Determination and monitoring of the position of the patient's head in the space in relation to the visual stimulator placed in front of the patient.
2) Determination and monitoring of the inclination of the patient's head in relation to the normal or vertical axis of the patient.
3) Control and monitoring of the rotation of the patient's head in relation to his axis.

The sensors determine and monitor the position, inclination and rotation of head; the algorithm processes the signals and generates instructions for the operator and/or patient as a feedback to keep the tests within the defined standard for each test.

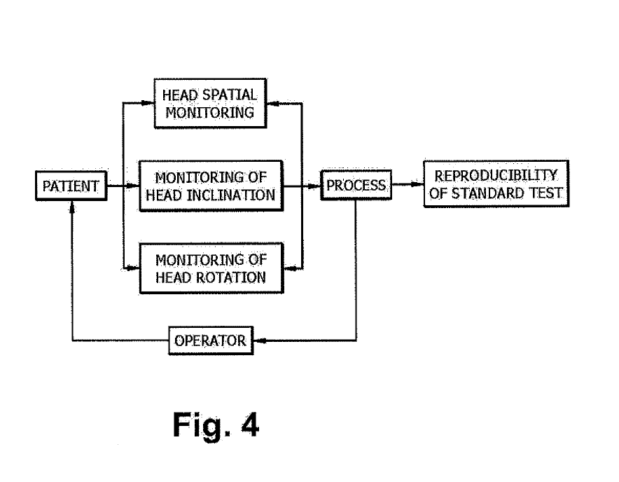

FIG. 4 shows a block diagram that displays the interaction between the operator and the patient during the VNG or ENG tests through the system of this invention.

In the most preferred way of realization of this invention, the portable system for the monitoring and positioning of the patient's head during videonysiagmography (VNG) and/or electronystagmography (ENG) studies, which allows the standardization and reproducibility of the said studies, is comprised of: nystagmus detection means; means for the determination in real time of the position and movements of the patient's head in space and their transmission through signals; a visual stimulator; and a personal computer (PC) connected to the nystagmus detection means, to the means for the determination in real time of the position and movements of the patient's head and to the visual stimulator, which has a software to determine the position of the eyes through the analysis of the data flow generated from the signals emitted by the nystagmus detection means; the position and movements of the patient's head in space through the analysis of the data flow generated from the signals emitted by the means for determining such position and movements; where the said means transmit those data flows to the PC; then the software compares the emitted signal with the signal received for each of the means used and calculates the position of the patient's head in space. Besides, the nystagmus detection means and the means for the determination in real time of the position and movements of the patient's head in space are arranged on the patient's head and in front of his eyes, while the visual stimulator is arranged before the patient's head and at height of his eyes, comprising also means for the detection of the spatial position of the patient's head.

In a preferred way of the system realization of this invention, the eyeglasses, which can also adopt the form of a mask or a helmet, contain at least two infrared chambers, with illumination of infrared light emitting diodes LED (LED's, acronym of Light-Emitting Diode/s) infrared, which fit in front of the eyes to permanently monitor the position of the pupil. Each chamber transmits a signal to the computer which calculates in real time the position of each eye. Over the eyeglasses an ultrasound transmitter is mounted, which generates an ultrasonic signal that it is detected by ultrasound receivers, which transmit such signal to the PC, that compares the signal emitted in relation to the one received by each receiver and calculates the position of the patient's head in space. This process is also carried out in real time.

Also mounted on the eyeglasses is a detector of the vector gravity, which is used together with the ultrasound signals to determine in real time the angular position and inclination of the patient's head.

The visual stimulator is placed opposite the patient and is used for the tests of optical stimuli follow-up on the part of the eyes, during the optokinetic tests.

A suitable software controls the PC generating instructions to the operator from the processes of images and positions of the patient.

In this way the operator may know if the patient is making the maneuvers within the established limits or if he must correct any parameter, position or instruction to the patient.

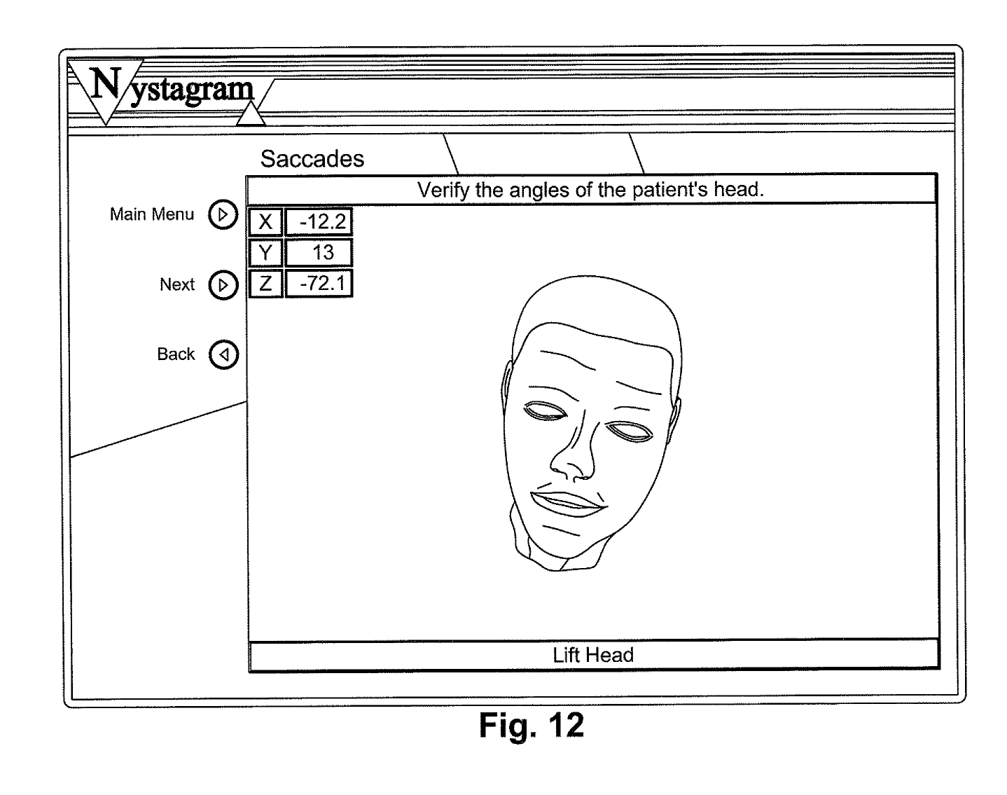

The PC's feedback to the operator consists of visual images of the position of the head and also in audible form, with verbal alarms and instructions. For example, see the screens that are shown in FIGS. 12 to 14.

The block diagram in FIG. 6 describes in detail the process of record, processing and transmission of information to the PC.

As for the patient, the system determines the position of the eyes by means of cameras, the three-dimensional position of the head by means of the ultrasound sensors and the angular position of the head by means of the detection of the gravity vector.

All these signals combine to generate the corresponding data flows. Such data flows are synchronized and modulated to be then transmitted to the computer via serial connection, for example, in parallel, of USB type, etcetera. Preferably, USB-type fast connections are used.

In the diagram block in FIG. 7 the process of signals carried out by the system is described.

The real time correlator device takes the preprocessed signals from the sensors and relates them to the limits imposed in the tests, which are those that determine the desired degree of reproducibility.

Also the target of the test is correlated, which is an input data that depends on each type of test.

The correlator sends the signal to the synchronization block that adds a base of time to the signals.

With these synchronized information it comes the analysis that has 3 parallel outputs: the first output is the real time analysis shown on the screen of the operator, the second is the subsequent process to store the information in the patient's file, and the third is the error estimation that compares the analyzed signal with the imposed limits.

This error triggers different outputs through the output generator which are divided into alerts for the operating technician and for the patient, through sounds caught by means of earphones, visual stimuli, audio for the operating technician and alarms.

The alarms are individual and distinctive for each type of violation of the limits previously fixed, like for example, alarm of angle, alarm of position, alarm of movement and alarm of follow-up of eyes.

In the oculomotor test visual stimuli are generated controlling the patient-stimulator distance, the movements of the patient's head, the angles of the patient's head, instructions are provided for the operator about the test technique and its conduction is controlled.

In the postural and positional test or DIX-Hallpike maneuvers, the angles of the patient's head in dynamic and static form are controlled, instructions are provided for the operator about the test technique and its conduction is controlled.

In the caloric stimulation test the static angles of the patient's head and the movements of the patient's head are controlled, instructions are provided for the operator about the test technique and its conduction is controlled.

In the rotatory test (AHR) the patient-stimulator distance, the movements of the patient's head and the angles of the patient's head are controlled, instructions are provided for the operator about the test technique and its conduction is controlled.

During the conduction of the tests, the system according to the present invention determines the angles and distances by means of ultrasound triangulation, infrared or laser devices and sensors. Gravitational sensors are capable of measuring the components of the gravity vector respect to the reference of the sensor and dynamic variations of acceleration.

The ultrasound detectors together with the gravitational sensors send their signals to a PC which processes the information by means of an algorithm that calculates the three-dimensional position and the speed of movement in real time of the patient's head.

The software includes two parts, the positioning algorithm which calculates the position and the speed, and the program that controls the VNG tests.

The VNG system software of the present invention, uses the information of the patient's positioning algorithm to generate alarms warning when the position and/or speed of the head is out the accepted range of the test that is being conducted.

The equipment rejects the results of badly made determinations and it only accepts the tests that are made within the admitted ranges, thus generating a safety net which ensures that the operator and the patient made the maneuvers of stimulation and taking of data in an adequate, reproducible and consistent way.

The feedback provided in real time in the form of alarms and visual guides, helps and guides the operator in the conduction of the tests.

The software is expected to have the capacity to generate images that guide the operator at any moment and whichever test is made.

FIG. 12 shows a screen that generates the Software verifying the static angular position of the patient's head in real time.

In this way the position of the patient's head can be verified and recommendations are provided for the operator:

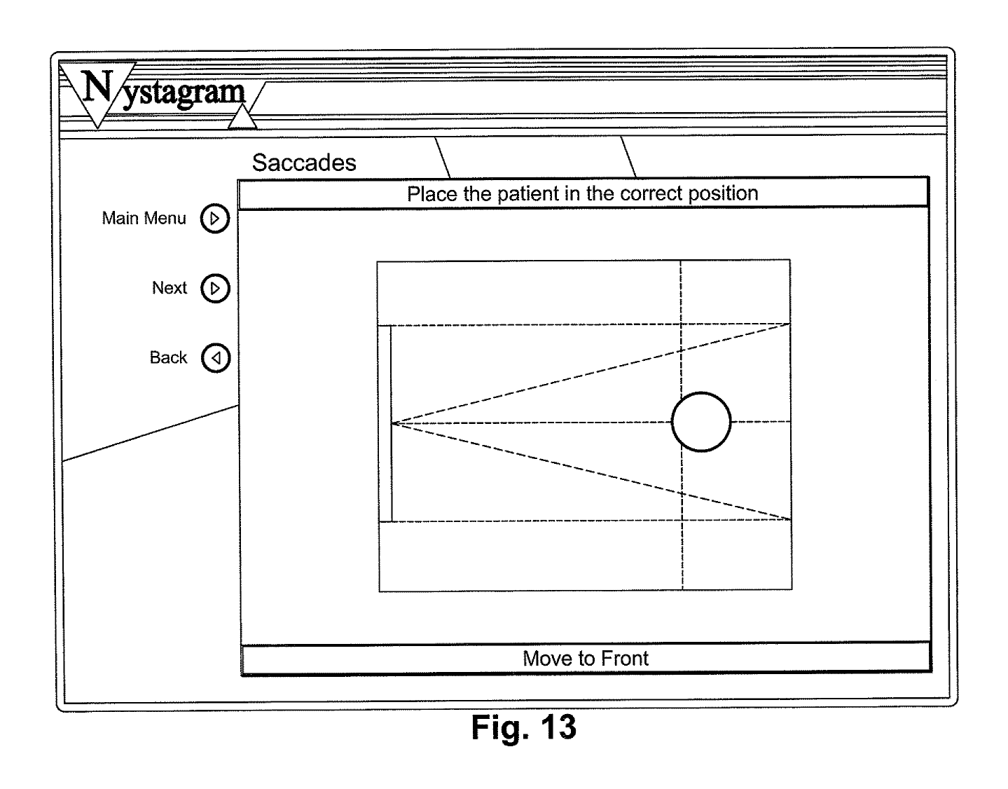

FIG. 13 shows a screen that generates the software that verifies the angular position of the patient's head in real time front of the visual stimulator. In case that the conduction of a test needs verification with correction suggestions of the patient's head positioning, software from the entering data can provide the corrections.

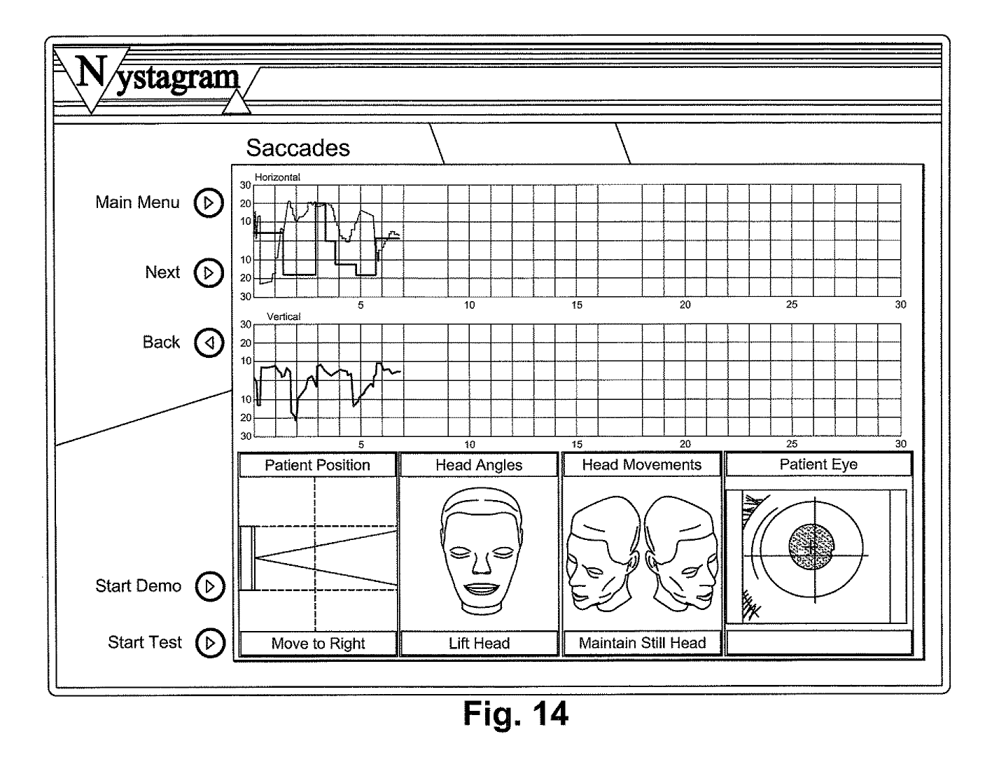

For example, the position control regarding the visual stimulator, the angular position of the head and the angular motion of the head, with alarm, for example in red, of movements or positioning outside the reproducibility range, along with the monitoring of the eye movement in real time, and a graphical representation of the eye movement in horizontal and vertical channels, can be obtained in a single screen as the shown in FIG. 14.

Everything described up to now can be modified in a clear way by someone who is expert on the art, without being necessary to stray from the scope of the claims accompanying this patent.

The claims are part of the description of this invention.

1. Portable system for the monitoring of a patient's head positioning during videonystagmography (VNG) and/or electronystagmography (ENG) studies, through which you can determine the position of a patient's head in relation to a visual stimulator placed before his head, the inclination of the patient's head in relation to his vertical axis (normal) and the rotation of the patient's head in relation to his vertical axis (normal), where the system re-feeds the said determinations of the position of the patient's head in space to the operator and/or patient with the objective of keeping the VNG or ENG studies within a defined standard that guarantees the reproducibility of those studies.

2. Portable system for the monitoring of a patient's head positioning during videonystagmography (VNG) and/or electronystagmography (ENG) studies standardizing and making it possible the reproducibility of the said studies, which is comprised of:
nystagmus detection means;
means for determining in real time the position and movements of the patient's head in space and transmitting them through signals;
a visual stimulator; and
a personal computer (PC) connected to the nystagmus detections means, to the means for determining in real time the position and movements of the patient's head and to the visual stimulator, which has a software that determines the position of his eyes through the analysis of the data flow generated from the signals emitted by the nystagmus detection means; the position and movements of the patient's head in the space through the analysis of the data flow generated from the signals emitted by the means that determine the mentioned position and movements; where those means transmit the said data flows to the PC, and then the software compares the emitted signal in relation to the signal received for each of the means used and calculates the position of the patient's head in the space;
wherein the nystagmus detection means and the means for determining in real time the position and movements of the patient's head in the space are arranged on the patient's head and before his eyes; while the visual stimulator is arranged before the patient's head and at the height of his eyes, comprising also means for the detection of the spatial position of the patient's head.

3. The system of claim 2, where the nystagmus detection means are infrared cameras arranged in a way that they are able to detect the position of the patient's pupil or electrodes arranged around the eyes of the patient.

4. The system of claim 3, where at least two infrared cameras, with infrared Light Emitting Diodes (LED), determine in real time the position of the patient's eyes through signal transmission; the said infrared cameras are adjusted in front of the patient's eyes.

5. The system of claim 2, where the means for determining in real time the position and movements of the patient's head in the space are comprised of at least one ultrasound transmitter that transmits ultrasonic signals.

6. The system of claim 2, where the means for determining in real time the angular position and inclination of the patient's head are comprised of at least a detector of gravity vector mounted on the patient's head, which transmits signals that are used together with the ultrasound signals.

7. The system of claims 2, 3, 4, 5 or 6, where the nystagmus detection means and the means for determining in real time the position and movements of the patient's head in the space and transmitting them through signals are arranged on the patient's head mounted on a pair of goggles, headband, mask or helmet.

8. The system of claim 2, where the visual stimulator is a set of lights arranged in a plane, a display of cathode ray tube or a liquid crystal display.

9. The system of claim 8, where the visual stimulator is comprised of a display that shows the images corresponding to the test and has at least three ultrasound detectors, one of which is arranged above the said stimulator and the other two, at both sides of it.

10. The system of claim 2, where the said data flows are synchronized and modulated for subsequent transmission to the PC through a serial connection.

11. The system of claim 10, where the serial connection is in parallel, or a USB type of connection.

12. The system of claim 11, where a USB type of connection is preferably used.

13. The system of claim 2, where the visual stimulator is comprised of four ultrasound receivers: one is mounted on the upper part, two are mounted laterally and the last one is mounted on the lower part of the said visual stimulator.

14. The system of claim 2, where the visual stimulator is used for monitoring tests of optical stimuli of the eyes during Optokinetic tests.

15. The system of claim 2, where the PC software generates instructions for the operator in the display, taking into account the data flow analysis of the image processes and positions adopted by the patient.

16. The system of claim 2, where the PC software provides the operator with visual images of the position of the patient's head and audible feedback.

17. The system of claim 16, where the PC software provides the operator with an audible feedback through alarms and oral instructions.

18. The system of any of the previous claims, through which you can carry out VNG or ENG studies that allow the exploration of the vestibular system of a patient.

19. The system of claim 18, where videonystagmography studies consist in the realization of at least a selected subgroup of tests such as Oculomotor or Optokinetic tests (Saccades, Smooth Pursuit, OKN, Gaze and Spontaneous Nystagmus); Postural and Positional tests (Positional and Positioning; Dix-Hallpike Manoeuvres); Caloric Stimulation; and Rotatory tests (Active Head Rotatory, AHR).

20. Portable system for the monitoring of a patient's head positioning during videonystagmography (VNG) and/or electronystagmography (ENG) standardizing and making it possible the reproducibility of the said studies, which is comprised of:
a pair of goggles comprised of at least two infrared cameras, with infrared light emitting diodes to determine in real time the position of the patient's eyes through signal transmission, where the said infrared cameras are adjusted before the patient's eyes; at least one ultrasound transmitter mounted on the said goggles to determine in real time the position of the patient's head in the space through ultrasonic signal transmission; and at least a detector of gravity vector mounted on the goggles to determine in real time the angular position and inclination of the patient's head through signal transmission that are used together with the ultrasound signals;
a visual stimulator comprised of a display that shows the images corresponding to the test and at least three ultrasound detectors, one of which is arranged in the upper part of the said stimulator and the other two, at both sides of it; and
a personal computer (PC) connected to the goggles and visual stimulator, which is comprised of a software that determines: the position of the eyes through the analysis of the data flow generated from signals emitted by the infrared cameras; the angular position of the head through the analysis of the data flow generated from signals emitted by the detector of gravity vector; and the three-dimensional position of the head through the analysis of the data flow generated from signals emitted by the ultrasound transmitter and detected by ultrasound receivers, which, in turn, transmit the said signal to the PC, where the emitted signal is compared to the received signal of each receiver, and where the position of the patient's head in the space is also calculated;
wherein, the goggles are arranged on the patient's head and in front of his eyes, while the visual stimulator is arranged before the patient's head at the height of his eyes.
